(19) United States
(12) Patent Application Publication
(10) Pub. No.: US 2023/0226276 A1
(43) Pub. Date: Jul. 20, 2023

(54) Self Injectable, Pre-Filled Syringes, Vials and Combinations Thereof

(71) Applicant: Ahmad Amiri, Richmond Hill (CA)

(72) Inventor: Ahmad Amiri, Richmond Hill (CA)

(73) Assignee: Inventall Inc., Richmond Hill (CA)

(21) Appl. No.: 17/580,280

(22) Filed: Jan. 20, 2022

(51) Int. Cl. A61M 5/24; A61M 5/46

(52) U.S. Cl. CPC A61M 5/24; A61M 5/46; A61M 2005/3114

(57) ABSTRACT

A syringe for injecting liquid medicine into a body, having its needle inside said syringe liquid holding barrel, pointed at one end of the syringe barrel which rests on user's skin when injecting, which end is sealed to hold the liquid inside the barrel of said syringe by a rubber like seal, through which seal said needle's sharp tip can exit when the needle is pressed towards the body via the other end of the barrel, which needle has conduit(s) near the other end of the needle to let said liquid into the inner tube of the needle to exit via said tip when the liquid is pressured as the needle is pushed into the user's body, which syringe can be prefilled during manufacturing, so that the syringe acts as a vial too, enabling self administration upon unpacking.

This application claims priority of US Provisional Applications 63/142,476 filed Jan. 1, 2021 and 63/143,850 filed Jan. 30, 2021

Specification:

This is to introduce various Syringes, Vials and or their combinations to provide numerous advantages, including safe and easy Administration (Injection) without the need for any health professional or anyone.

Pre-Fillable Sealed Syringe—A Syringe, one version shown in FIG. 1, which is Pre-Filled (FIG. 1-1), by manufacturer, supplier, user or patient, from a bulk container, with one or more doses of a medicine, which medicine fills also the syringe's Needle.

Said needle is protected by an Optional Cover (FIG. 1-2), along some to all of said needle's length or more. The needle's Tip (Bevel) is sealed by a Cap (FIG. 1-3), preventing contact with and entry of external air into the needle and leakage of the medicine out. One option for the cap and or the said cover is a Rubber segment, into which said tip is inserted to provide said sealing. Said cap and cover can be one integral component, but even though a good material for the cap is rubber or rubber likes such as silicones, the length of the needle better be protected by a rigid or semi rigid material such as plastic. So a plastic Tubelike Cover with one end open for needle entry and the other end a rubber acting as said Cap is a good structure. Said cover, better have Latching means (FIG. 1-4) to removably attach its open end to the rest of the syringe to prevent unintended separation form the syringe.

During Filling the syringe with medicine, the syringe Plunger is positioned in full contact with medicine such that insignificant or No Air is left in the syringe and needle. The syringe barrel has Volume Markings (FIG. 1-5) to show how much medicine is inside, so that for example one filling can be divided into more than one doses.

The Plunger can have Fixing means to prevent unintended plunge or move. One method is to use a removable adhesive tape/patch (FIG. 1-6), known to the skilled, which may also show if any tampering has occurred.

Said Assembly better be held inside a suitably designed package for transport, storage and handling.

This version suits a user who can inject herself, or has access to someone who can. But saves a vial, medicine and time waste for filling, de-airing and dosage accuracy. Simply remove the cover, cap and any fixing to inject, stop when enough for one dose is injected, replace the cover, cap and any fixing until the next injection. An unqualified intern can do hundreds of fillings and store them for a nurse.

Above introduces at least a Needle Cap, Sealed Syringe and a Method of easing and speeding injections.

Pre-Fillable Syringe Vial—A Syringe with Vial Functionalities as in FIG. 2 which is self explanatory.

The Needle (FIG. 2-7) has a Base (FIG. 2-5) placed inside a Well dug into the Barrel's (FIG. 2-8) Lid (FIG. 2-17). If the needle should not unintentionally dislodge out of the well, said base can be Screw like, held in place by friction against well's walls, glued in place, or use other means known to the skilled. Said base can have any of various cross sections, but circular may allow some unintended rotation of the needle. The base should be long enough and the well deep enough, tightly holding the base to ensure the needle's Tip does not move laterally, and remains pointed towards the barrel's Liquid Outlet (Needle Exit Hole FIG. 2-12). The Lid is made of solid or semi solid material, at least around said well to ensure needle's stability. But may have Rubber or Rubbery material touching the inner wall of the barrel to seal the liquid inside the barrel.

The Needle has a Waist section (FIG. 2-6) with Conduit(s)/Hole(s) for the Liquid to enter the needles hollow Core for exit from needle's tip. Said waist may have a larger diameter than the rest of the Tube segment of the needle (Shaft), to enable higher liquid flow, and because holes around the needle's tube without the wider waist may lead to easy bending of the needle's tube. If the needle's base is of screw version, the waist's cross section can be non circular to be used for screwing the base in and out of the well.

The needle's base, waist and tube can be separate components but to avoid costs and complexities, an integral construction is often preferred.

The needle's Tip (FIG. 2-13) is inserted into, but not extruded out of, a rubber or rubber like Cap (FIG. 2-11), (also called a Seal), which cap is adhered to the inner Bottom of the Barrel, firm enough to prevent medicine leakage. The cap better extend and press firmly against the barrel's inner walls, rather than bottom, potentially saving adherent or glue for said leakage prevention. The cap better have a preferably smooth, solid and or slippery needle tip Guiding Funnel (FIG. 2-10), the narrow end of which Funnel encircles the inserted needle closely to limit needle wobble or tilt.

Needles with different Lengths and or Diameters can be used and interchanged.

The bottom of the barrel has a Needle Exit Hole to enable needle's tip to exit the barrel. Said hole is sealed by the Rubbery segment of the cap, which rubber allows the needle tip out of the barrel and closes in when the needle is removed. Said bottom can be detached from the barrel and attached to the cap of the type that extends to the barrel's inner walls, forming a removable cap that acts as the barrel's bottom too, in which case the cap better have a solid or semi solid external layer, replacing said barrel bottom layer, to resist bulging out when the medicine is under plunger pressure.

The Contour of inner surface of the barrel's bottom with said cap in place better match the contour of the inner surface of the lid, such that when said inner surfaces are in full contact, the least amount of medicine is trapped without exiting the barrel, to minimize waste.

The Plunger's Handle (FIG. 2-1) can be conventionally attached to the top (external surface) of barrel's lid, which lid also acts as a conventional plunger's seal inside the barrel. But said handle is not essential, as a finger or any rod like apparatus can be used to push said lid down (plunge). Said handle, if attached to the lid, may cause unintended pressing of the lid. One preferred version is that said handle is detached from but placed over the exterior top of the lid to press the lit into the barrel. Preferably, the handle should latch onto the lid (FIG. 2-3), using any of many known means, such as screwed in, to enable pulling the lid up after plunged in, even out for one method of refilling. Another way of enabling lid pull up and or out is to attach a Finger Ring hinged to or a String attached to the top of the lid. One handle can be used for many different syringes, each many times, thus no need to accompany a handle to each barrel.

Refilling can be done by removing the lid or the barrel bottom if removable. Another way is via Filling Hole(s) (FIGS. 2-2 & 18) through the barrel bottom and or lid, which holes are sealed by a Rubber or Rubber like Layer within same bottom or lid, such that a supply syringe is inserted via one of said filling holes, through said rubbery layer, to replenish the medicine, then pulled out, as the rubbery layer closes the syringe's piercing.

For De-airing, the Contour of the lid inside the barrel can have at least one Concave Dip close to one of the filling holes. A conventional syringe needle tip is inserted via said nearby hole barely exiting the rubber layer, into said dip, letting excess air (FIGS. 2-16) out, due to filling pressure. Said dip can also be carved onto the inner surface of the bottom cap connecting to a corresponding filling hole.

The barrel need not have a circular cross section. In fact a rectangular one has more capacity than a circular one of same diameter and easier to hold.

Barrels bottom better be flat, to rest on the skin, with less risk of skidding when the plunger is pressed, keeping the needle perpendicular to the skin. But an outwardly Convex barrel bottom eases resting the barrel non perpendicular to the skin, if non-perpendicular needle entry into user's body is desired. Convex barrel bottom can be smooth, for a continuum of barrel to skin angles, may have multiple planar surfaces, each plane easing a certain angle or have both types of convex surface bands which bands cross each other, enabling both continuous and discrete angles of barrel to skin.

The barrel can have no, small or large flange(s) (FIG. 2-4). A small flange can be supplemented by an external flange, being a ring with flange(s) tightly surrounding the barrel, which barrel is run through said ring, until the flanged ring meets the barrel's small flange(s).

The syringe barrel has Volume Markings (FIG. 2-9) to show how much medicine is inside, so that for example one filling can be divided into more than one doses.

Above enables a single injection of a dosage depending on needle length and barrels cross section area. So for a large dose and short needle, a larger cross section is needed and vice versa.

For a second dose capability, a longer needle can be used, part of which exits the barrel after the first injection, then a Barrel Extender as in FIG. 3 with rigid walls (FIG. 3-3) and almost the same cross section as the barrel, with an opening to tightly encircle the lower rim and or walls of the barrel, a cap at the opposite end inside the barrel extender, replicating the cap inside the barrel, to receive needle's tip can be inserted. Second injection is performed by placing the barrel extender's bottom over the skin and plunger pressed. Ditto for next injection(s).

Injecting—Is done by placing the bottom of the barrel over the skin and pressing down the plunger by one hand, preferably holding the barrel by the other, which is possible on most injection sites. For upper arm, either one hand injection is performed or another person may hold the barrel. No expertise required.

When Self Injection is not easy for a person, any non expert can do the injecting.

Pre-Filled Syringe Vials can be distributed by a local pharmacy or shop, administered there upon purchase, even from over the garment, by the user, shop assistant or by-passer.

Syringe Extension Barrel

This Invention introduces Aiding Accessory(s) and or Assemblies for easier or Self Injection of both Pre-Filled and site filled Syringes, especially if it is desired that the needle is inserted to at least some depth before injection I dispensing of medicine, one shown in FIG. 4.

A Syringe Barrel Extension's Inner Walls (FIG. 4-5) tightly surround the outer walls of the barrel. The bottom of the Extension is placed over the skin, the barrel is pressed down, preferably by its flange(s) (FIG. 4-2), and only when in its desired depth, the plunger is pressed to release the medicine.

Especially for the Pre-Filled Syringe, the needle's Tip is inserted into a Rubber or Rubbery Sealing Cap, (FIG. 4-7) which can be made removable or pierced by the needle before injection.

Any of many known Means of holding/preventing the barrel from unintended moving into the Extension can be added, one is a Hold Plug (FIG. 4-13) inserted in a Hole on the Extension side, to be removed before injecting.

Any of known Means of stopping the barrel's movement into the Extension can be added, one shown as Stop Plug (FIG. 4-6), inserted into one hole, one of many disconnected holes, one of many connected holes (shown), a continuous opening for fixing the Stop Plug anywhere along its length, and or a combination of said versions. Said Stop Plug holes or opening need Not be along a vertical or straight line. If desired, longer Stop Plug Entry lines can be produced curved, waved, snake, zig zag and or other. Stop Plug can be used also as Hold Plug, then repositioned as Stop Plug.

Hold plug and stop plug can be attached via a Tail (FIG. 4-12) , especially to the multi use Barrel Extension.

The Barrel Extension need Not have a Conical Bottom, but can have Convex (Dome), Multi Planar Convex, combination Convex Smooth Band and Multi Planar Band, crossing each other. One good version is a Flat Bottom, to better rest on the skin, especially for needle perpendicular injection into the skin.

Any of many known Barrel Extension Fixing Means to prevent the barrel from unintended exit from the Extension can be used, such as a Sticking Tape/Patch, known to the skilled, ash shown (FIG. 4-14), which can have tamper exposing or alert means too.

A Syringe Stabilizer can be added, as shown (FIG. 4-10). A rigid or preferably semi rigid pad with non-slippery underlayer material , such as rubber, silicone, or other, that rests on the skin (FIG. 4-9), preventing skidding of the Assembly's needle exit over the skin.

Said stabilizer is connect to the needle exit via a Needle Guide (FIG. 4-8), being a Hollow Hinge, a simple version of which is a rubber or rubber like tube, preferably not too soft, not to collapse, but flexible to enable the Extension Barrel and the rest of the assembly to be held in various directions against the skin, such as almost parallel for injection under the skin to vertical for deep muscular injections.

Accessories to Ease Syringe Injections:

Syringe Stabilizing Base—If the Syringe bottom is too small to rest stable on the skin, a tube with a wider base (FIG. 5-A-1) can be used, to hold the barrel, barrel extension or extender (FIG. 5-A-4) over the skin (FIG. 5-A-2). One hand holds the barrel or the stabilizer for the other hand to do the rest of injecting. Said base better have a non slippery, such as Rubber underlayer that touches the skin. The base can be integral to Syringe.

Syringe Needle Direction Guide & Stabilizer—An Inverted Cone or similar shape to suit the syringe it will serve (FIG. 5-B-5), with a Base (FIG. 5-B-2) that rests on the skin (FIG. 5-B-3). Said Base & Cone are connected via a Multi Directional Hollow Hinge, through its hollow joint (core), the needle (FIG. 5-B-7) can pass to penetrate the skin. Said hinge, can hold the syringe and or guide the needle in the desired angel towards the skin. Its range can be from a narrow and limited to full x-y plane or limited or full all directions. Said Hinge better have some resistance to pivoting in one or any direction to stay reasonably motionless in the desired direction while injecting. The base better have a non slippery, such as rubber underlayer not to skid over the skin.

An alternative is to add a Cylinder (FIG. 5-B-6) to the Cone, to hold the barrel or extension(s).

One such Hollow Core Hinge is shown, two concentric abridged globes (FIGS. 5-B-1 & 4) that can slide against each other. At least the outer shell better be larger than a semi globe (semi plus) to ensure the inner one does not fall out of it. Any of many other hinges that perform above functions, known to the skilled, can be used.

Body, Muscle, Skin Grab Jaws—For grabbing the skin, body fat and or muscle as desired, a device as in FIG. 5-C is introduced. Two conjoint Jaws (FIG. 5-C-1), movable towards each other by spring action or from their joint Hinge (FIG. 5-C-5), while a Loop (FIG. 5-C-4) that can be Locked when the required body mass (FIG. 5-C-3) is grabbed. The Jaws have non-slippery linings such as rubber (FIG. 5-C-2), to better hold the skin around the grabbed body mass.

One situation is for upper arm that better be grabbed, when self injecting, as the injected arm's hand cannot be used to grab, while the other hand is injecting. Another is to hold belly skin and or fat for insulin shots.

Any of many known Lock Means can be used.

Straps—Elastic, cloth or other straps can be attached to the Syringe Stabilizer via Means attached to the strap, such as Grabs, Jaws, Claws, Pins, . . . , Means on the Stabilizer such as Slits, Holes, Loops, Rings, . . . and or Means on both such as Velcro, to attach straps to the stabilizer and tighten them to the body via Ties, Buckles, Fasteners, and so on. For example, a strap can secure a stabilizer placed on the upper arm, to around the neck and another strap can secure it around the arm. A wrist band can secure a stabilizer to he wrist or arm.

Practice Dolls—Rubber or Sponge versions can be used for practice by water filled Syringe assemblies.

Independent Needle & Medicine Plungers—Of use especially for Pre-Fillable Sealed Syringe, when it is desired that the needle penetrates the users body to some depth before the medicine is injected, the needle can have its own plunger (FIG. 6-5), operating independently of the liquid plunger (FIG. 6-8). The needle is connected to its own plunger handle (FIG. 6-4) which runs through a sealed Core tube in the Vial and or Syringe Medicine Sealing (FIG. 6-3), which core's walls firmly surround the needle plunger's handle, such that the medicine cannot leak through said core tube. For example at least the inside of the core tube is or is lined with rubber or rubber like material (FIG. 6-2), or is of well polished glass. Also the needle plunger handle is of polished exterior.

The needle plunger can have a removable handle, with a Pin (FIG. 6-1), as shown, that enters a matching well inside the needle's Base.

The needle plunger is alongside the medicine plunger, preferably inside the medicine plunger's handle's which for this purpose should be a hollow core, while the body of the medicine handle has vertical gaps or openings (FIG. 6-7) to let the needle's plunger Flanges (FIG. 6-6) out and accessible by a user's fingers and thumb.

Thus, the needles plunger can be pressed down first, until the needle penetrates to desired depth, then the medicine plunder is pressed to inject the liquid.

### BRIEF DESCRIPTION OF DRAWINGS:

FIG. 1 shows Pre-Fillable Sealed Syringe

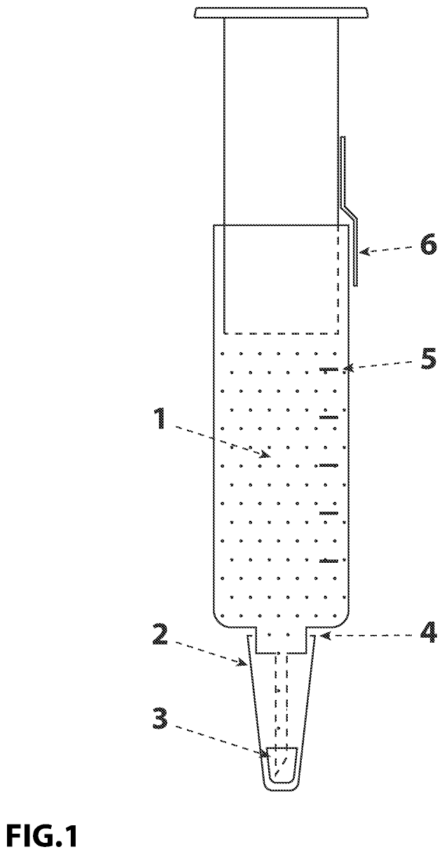

FIG. 2 shows a Syringe with Vial Functionalities

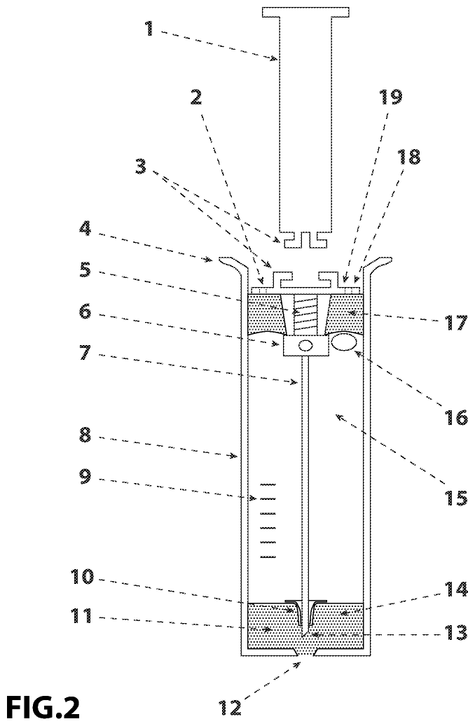

FIG. 2A shows a blown up of the upper part of the syringe in FIG. 2

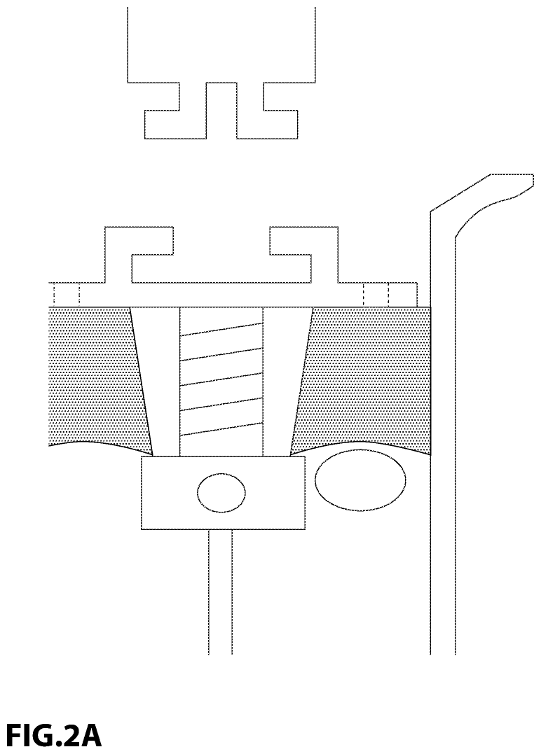

FIG. 2B shows a blown up of the lower part of the syringe in FIG. 2

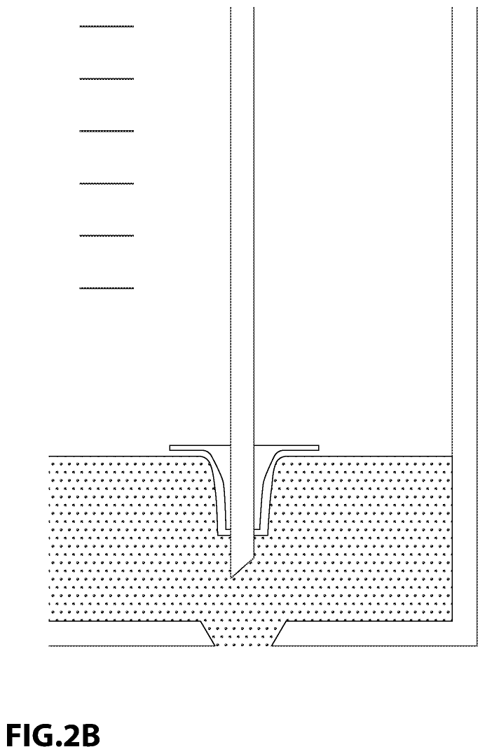

FIG. 3 shows a Syringe Barrel Extender

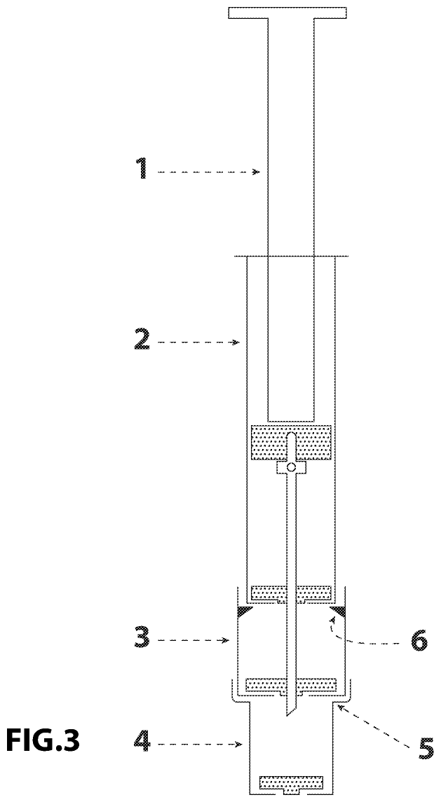

FIG. 4 shows a Syringe Extension Barrel

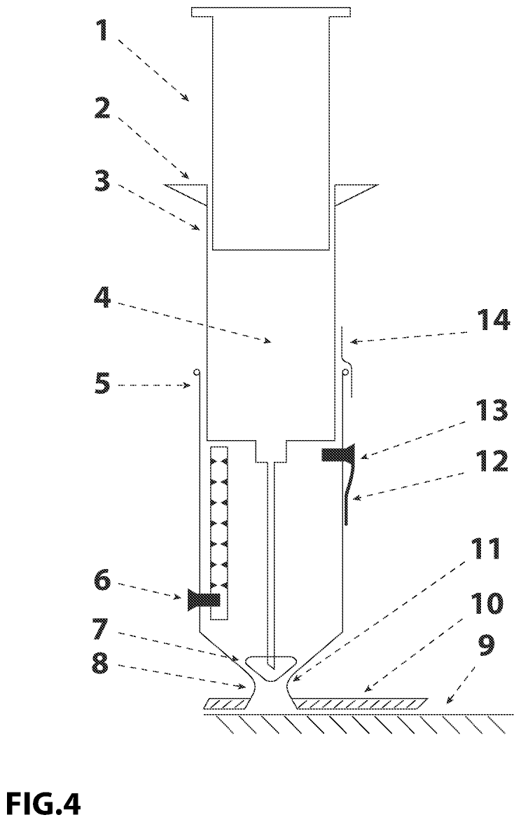

FIG. 5-A shows a Syringe Stabilizing Base

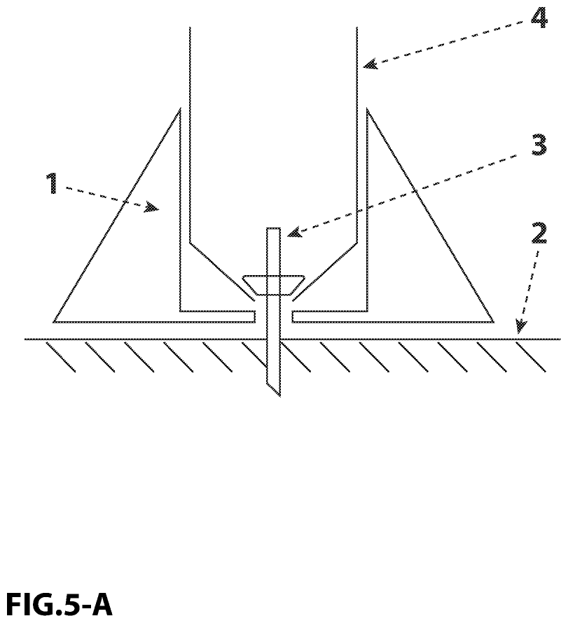

FIG. 5-B shows a Syringe Needle Direction Guide & Stabilizer

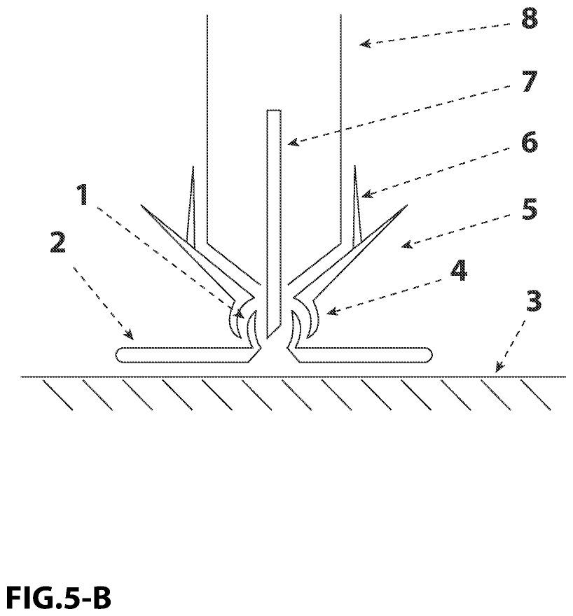

FIG. 5-C, shows a Body, Muscle and or Skin Grab Jaws

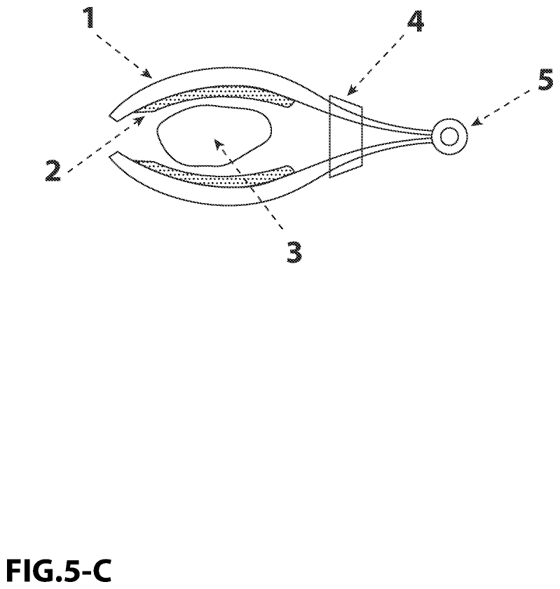

FIG. 6 shows Independent Needle & Medicine Plungers

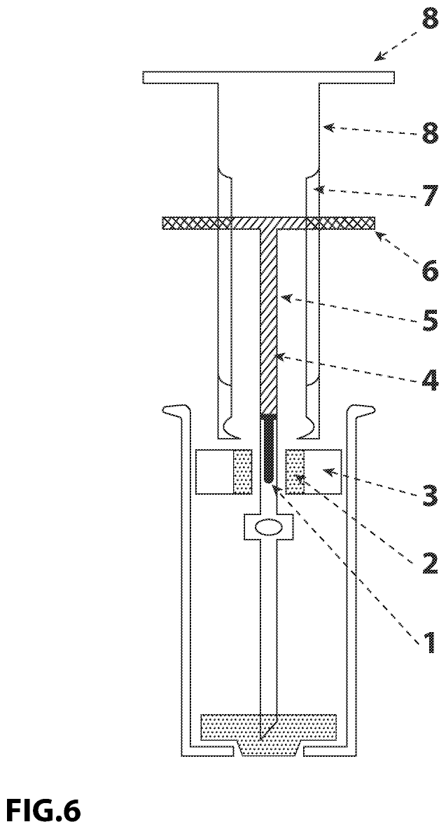

Some Clarifications (including some terms used in claims):

Needle Adapter used in conventional Syringes can be modified to be as the Needle Base introduced here.

Needle Hub uses in conventional Syringes can be modified to be as the Needle Waist introduced here.

The part of Plunger used in a convention Syringes which is adjacent to the liquid, to be called plunger head here, can be modified to act as the Lid introduced here, to harbour the Needle's Base introduced here.

Needle's hollow tube is also called Shaft.

Needle's sharp tip is also called Bevel.

The Syringes and other inventions introduced here can be made for non medical uses too.

The Syringe Extension Barrel can be used for both conventional syringes and those introduced here.

The Barrel's Liquid Outlet is a hole at the Barrel's Bottom via which the liquid may exit.

1. A syringe, including a barrel with an opening at one end and a liquid outlet at its other end, wherein, in pre injection mode:
said barrel contains a lid, at least part of said lid is within said barrel's interior,
the entirety of said lid's circumference is tangential to the inner surface of said barrel, tightly enough to resist leaking of any liquid out of the barrel from around said circumference,
said lid is slidable along the interior of said barrel, between said barrel's opening and outlet,
said lid harbours a syringe needle, which shaft is extended off the lid, inside said barrel,
the sharp tip, a.k.a bevel of which needle is pointed towards said barrel's outlet,
said needle's end, other than tip, called the base here, is firmly attached to said lid,
said lid has means to secure the needle's base and maintain its tip towards said outlet,
said needle has a liquid inlet at a segment of the needle called waist here, positioned substantially close to said base, allowing the liquid from the barrel into the needles core tube,
said barrel's liquid outlet is blocked by a seal to prevent liquid escaping the barrel,
and in addition to all above, said seal is penetrable by the needle's tip,
thus, when said lid is pressed into the barrel, as the exterior of the barrels outlet end is resting on a patient's body, said needle's tip pierces said seal, then the patient's skin, while said liquid is pushed via said needle's inlet, through the needle's hollow core to needle's tip and released inside said body, whereby numerous advantages over conventional syringes are achieved, some being:
said lid can be pushed towards the barrel outlet using a finger or rod, as a plunger's handle,
the syringe can be self administered, no skill needed to hold the needle at proper distance from and angle with a patient's skin,
the needle is held by both said lid and seal, before being pushed into the user's body, providing more stability and less chance of bending of needle before and after entering the body,
liquid is dispersed along all of needle's travel inside the body, providing much better absorption, compared to conventional release at the end point of needle's travel,
the needle, is not visible by the patient, easing anxiety,
no chance of the needle being accidently touched or damaged before administered,
the needle is protected by the barrel and needs no protection by costlier packaging,
the syringe is much shorter for less packaging volume and material, and many other advantages.

2. Syringe of claim 1 wherein said lid has a handle extending towards barrel's opening, whereby the handle aids maneuvering said lid.

3. Syringe of claim 1 wherein said lid has a handle extending towards barrel's opening, which handle is disjoint from said lid, whereby one handle can serve more than just one syringe.

4. Syringe of claim 1 wherein said lid has a handle extending towards barrel's opening, which handle is disjoint from said lid, said handle and lid have means for latching to each other, whereby said handle can be used to pull the lid away from the barrel's outlet, whereby the syringe can be pulled back into the barrel, for safety and to enable filling and refilling the barrel via the needle.

5. Syringe of claim 1 wherein said lid has a handle extended towards barrel's opening, which handle is disjoint from said lid, said handle and lid have means of latching to each other by screwing one into the other, whereby said handle can be used to pull the lid away from the barrel's outlet, whereby the syringe can be pulled back into the barrel, for safety and to enable filling and refilling the barrel via the needle.

6. Syringe of claim 1 wherein said lid has means for pulling it away from barrels outlet, whereby the syringe can be pulled back into the barrel, for safety and to enable filling and refilling the barrel via the needle.

7. Syringe of claim 1 wherein said lid has a ring on its surface accessible form barrels opening, which ring can be used by a finger for pulling said lid away from barrels outlet, whereby the syringe can be pulled back into the barrel, for safety and to enable filling and refilling the barrel via the needle.

8. Syringe of claim 1 where said needle's tip rests inserted into said seal.

9. Syringe of claim 1 wherein a funnel is inserted into said seal, which funnel's inlet is positioned to receive said needle' tip, said funnel's outlet remaining within and sealed by said seal, pointed towards said barrel outlet, whereby the needle is guided via the funnel towards the outlet.

10. Syringe of claim 1 wherein the mechanism of attaching said needle's base to said lid has means to enable replacing the needle.

11. Syringe of claim 1 wherein any layer of said lid which cannot be penetrated by a needle has at least one hole through which the needle of another syringe can be inserted into the barrel, whereby the barrel can be filled, emptied and de-aired.

12. Syringe of claim 1 wherein any layer of said lid which cannot be penetrated by a needle has at least one hole through which the needle of another syringe can be inserted into the barrel, the surface of said lid facing the liquid has at least one concave dip where said needle tip penetrates said surface, whereby the barrel can be maneuvered to collect any air into said dip to be de-aired by said other syringe.

13. Syringe of claim 1 wherein the surfaces of said lid and said seal that face each other are contoured for maximum mutual contact when they meet, whereby least volume of liquid can be trapped between them when said surfaces are in full surface contact.

14. Syringe of claim 1 wherein the outer surface of said barrel facing a patient's skin when injecting is concave, whereby resting it on the skin and holding it at a desired angle is eased.

15. Syringe of claim 1 wherein the outer surface of said barrel facing a patient's skin when injecting is overall concave with a number of planar surfaces, whereby resting said one of the surfaces on the skin and holding it steady at an angle determined by said surface is eased.

16. Syringe of claim 1 wherein said barrel is prefilled and de-aired, ready to inject, whereby much time, skill, liquid waste, inaccuracies and risks of onsite filling and de-airing is saved, enabling buying from a local shop and self injection by even a child.

17. Syringe of claim 1 wherein said needle has its own means of plunging it into a patient's body, as said lid remains unmoved, to be pressed to release the liquid, after the needle has entered desired depth inside a patient's body.

18. Syringe of claim 1 wherein said needle's base has a hollow core, called a well, opened at the lids surface facing the barrel's opening, into which well a rod can be inserted, via the barrel's opening, said base is slidable off the lid when pushed by said rod, said rod's exterior which remains outside of the needle's base has a diameter and shape matching those of the interior of said tunnel, sufficient to prevent liquid leakage when the rod is pushed into the barrel, whereby said needle can be inserted to desired depth into a patient's body, before said lid is pushed to release the liquid.

19. Syringe of claim 18 wherein said rod has means to latch with the needle's base, whereby the needle can be pulled back into the barrel.

20. Syringe of claim 18 wherein said rod is screwed into the well, as means to latch with the needl's base, whereby the needle can be pulled back into the barrel.

21. Syringe of claim 18 wherein said rod branches into more than one rods at the end which is out of the needle's base, said lid has a hollow core handle whose shell like body surrounds said rod, said shell via which said branches exit enough for a finger to contact and push or pull the branch, whereby the rod can be pushed into and pulled out of the barrel, while the lid plunger handle remains unmoved.

22. Syringe of claim 1 wherein at least one of said needle's base and waist are detachable from the needle and replaceable.

23. Syringe of claim 1 adapted for at least one non medical application.
